(19) United States
(12) Patent Application Publication
(10) Pub. No.: US 2018/0268669 A1
(43) Pub. Date: Sep. 20, 2018

(54) Emergency Responder Alert System

(71) Applicant: Rocco Lucciola, Brick, NJ (US)

(72) Inventor: Rocco Lucciola, Brick, NJ (US)

(21) Appl. No.: 15/974,261

(22) Filed: May 8, 2018

Related U.S. Application Data
(63) Continuation-in-part of application No. 15/457,112, filed on Mar. 13, 2017, now abandoned.
(60) Provisional application No. 62/322,332, filed on Apr. 14, 2016.

(51) Int. Cl. G08B 7/06; G08B 25/01; G07C 9/00; A61K 31/616

(52) U.S. Cl. CPC G08B 7/064; G08B 25/016; G07C 2009/00769; A61K 31/616; G07C 9/00309

(57) ABSTRACT

An emergency responder alert system. The emergency responder alert system includes a housing having a container affixed thereto, wherein the container includes a bottom, a sidewall, and an open end defining an interior volume. A power source is connected to the housing and provides power thereto. The system further includes indicia having a fastener on a rear side, wherein a front side thereof is reflective and a light source to illuminate the indicia. A speaker is disposed on the housing and is connected to a microprocessor disposed therein. The microprocessor includes a logic that activates when a control on the housing is actuated. The logic contacts emergency services to allow two-way communication via the speaker, disengages a door lock mechanism, illuminates the light source, and sends an alert to a pre-determined third-party.

### CROSS REFERENCE TO RELATED APPLICATIONS

This application is a continuation-in-part of U.S. patent application Ser. No. 15/457,112 filed on Mar. 13, 2017 and claims the benefit of U.S. Provisional Application No. 62/322,332 filed on Apr. 14, 2016. The above identified patent application is herein incorporated by reference in its entirety to provide continuity of disclosure.

### BACKGROUND OF THE INVENTION

The present invention relates to an emergency responder alert system. Specifically, it relates to an emergency responder alert system having a housing and a container having aspirin therein, the housing including telecommunications means for communicating with emergency services in the event of an emergency, such as a heart attack.

Guidelines have been issued to help people to know what to do in the event that they or someone they know is having a heart attack. These guidelines instruct that the person suffering a heart attack take one chewable 325 mg uncoated aspirin tablets and then immediately call for emergency assistance, such as 911. However, a person who is at risk of having a heart attack may not have immediate access to aspirin at all times. This is particularly true for elderly individuals who may have difficulty moving about to locate aspirin or who may have poor vision that prevents them from identifying a bottle of aspirin. Further, according to several heart associations, the rate of heart attacks nationwide is approximately 790,000, leading to 114,000 fatalities. Thus, a device for allowing users to have immediate an easy access to aspirin in the event of a heart attack is desired.

Calling for emergency assistance is crucial when an individual is having a heart attack. However, some residences are not clearly marked such that an emergency responder knows exactly where to go. If an emergency responder passes the location to which they are dispatched, they have to turn around and locate the address. This wastes crucial time in which the individual may not have a moment to spare. Therefore, there further exists a need for a system and method of alerting an emergency responder of an exact location in which they have been dispatched. Specifically, a light source and address numbers designed to reflect light from an oncoming vehicle.

In light of the devices disclosed in the known art, it is submitted that the present invention substantially diverges in design elements from the known art and consequently it is clear that there is a need in the art for an improvement to existing emergency responder alert systems. In this regard, the instant invention substantially fulfills these needs.

### SUMMARY OF THE INVENTION

In view of the foregoing disadvantages inherent in the known types of emergency responder alert systems now present in the known art, the present invention provides an emergency responder alert system wherein the same can be utilized for providing convenience for the user when alerting emergency responders of an exact location in which they have been dispatched, which can save lives in the multitude.

The present system comprises a housing having a container affixed thereto, wherein the container comprises a bottom, an open end, and a sidewall therebetween defining an interior volume. A power source is operably connected to the housing such that the power source is configured to provide power thereto. The system further comprises indicia having a fastener on a rear side thereof and a reflective surface on a front side thereof, wherein a light source is configured to illuminate the indicia. A speaker is disposed on the housing, wherein the speaker is operably connected to a microprocessor disposed within the housing. A control is disposed on the housing, wherein the control is operably connected to the microprocessor. The microprocessor comprises a logic thereon, such that when the control is actuated, the logic is configured to contact emergency services, wherein a user can engage in two-way communication therewith via the speaker, actuate a door lock mechanism to disengage the door lock mechanism from the door, illuminate the light source, and send an alert to a predetermined third-party. In some embodiments, the power source comprises a battery, while in other embodiments, the power source comprises a home power line or a solar panel. In another embodiment the control further comprises a light thereon, the light configured to illuminate the control. In other embodiments, the control comprises a pull-cord. In yet another embodiment, the light source comprises a central light and a pair of lateral lights, wherein the central light comprises a diameter greater than a diameter of each of the pair of lateral lights. In some embodiments, the light source is configured to strobe. In another embodiment, at least one 325 mg uncoated aspirin is disposed within the interior volume. In other embodiments, the container further comprises secondary indicia thereon, the secondary indicia configured to instruct the user to take one 325 mg uncoated aspirin. In yet another embodiment, the system further comprises a cap configured to cover the open end. In some embodiments, a desiccant is disposed within the interior volume and affixed to the bottom of the container. In another embodiment, the container tapers outwards from the bottom towards the open end. In other embodiments, the front surface comprises a convex shape. In other embodiments, the cap further comprises a handle thereon. In yet another embodiment, the cap further comprises a lip extending perpendicularly away from a lower surface of the cap, the lip configured to removably secure within the open end via friction fit. In some embodiments, the container is affixed to a platform extending from the housing.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the housing of an embodiment of the emergency responder alert system.

FIG. 2 shows a cross-sectional view of the container of an embodiment of the emergency responder alert system.

FIG. 3 shows a perspective view of the lights and indicia of an embodiment of the emergency responder alert system.

FIG. 4 shows a schematic view of an embodiment of the emergency responder alert system.

### DETAILED DESCRIPTION OF THE INVENTION

Reference is made herein to the attached drawings. Like reference numerals are used throughout the drawings to depict like or similar elements of the emergency responder alert system. The figures are intended for representative purposes only and should not be considered to be limiting in any respect.

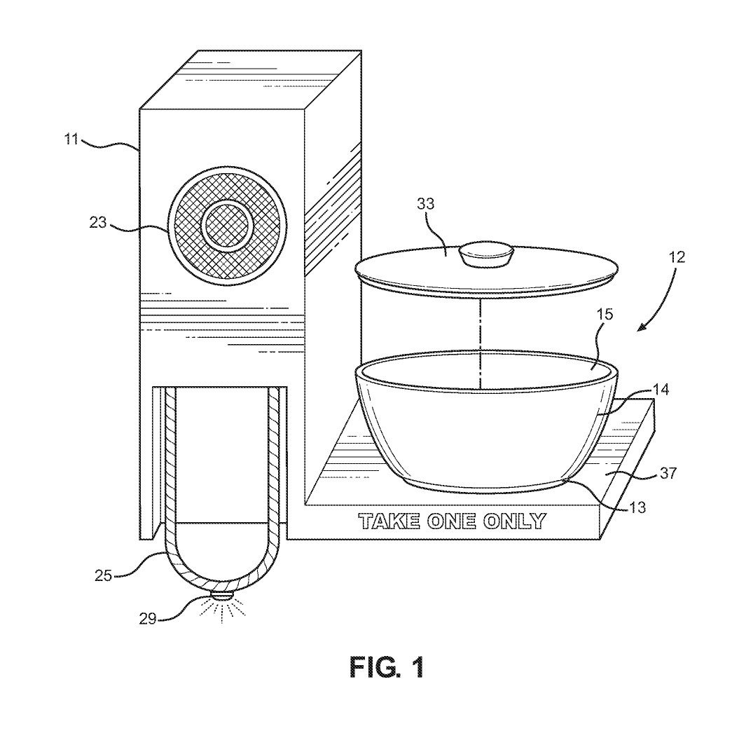

Referring now to FIG. 1, there is shown a perspective view of the housing of an embodiment of the emergency responder alert system. The emergency responder alert system comprises a housing 11 having a container 12 affixed thereto. The housing 11 is configured to be installed into a support surface, such as a wall, such that the housing 11 is readily available in an emergency. In one embodiment, the housing 11 is installed adjacent to a door, such that when the user feels chest pain indicative of a heart attack, the user can utilize the emergency responder alert system while being in position to receive an emergency responder into their home. In the illustrated embodiment, the container 12 is affixed to a platform 37, wherein the platform extends perpendicularly away from the housing 11. However, in alternate embodiments, the container 12 is directly affixed to the housing 11, wherein the housing 11 does not include the platform 37, such that the housing 11 comprises a smaller form factor allowing the housing 11 to be easily installed in a variety of locations. The container 12 can also be disposed within the housing 11, such that the container 12 is accessible via an opening therein. In yet another embodiment, the container 12 can include a slot therein, wherein the container 12 is configured to dispense a pill therethrough. In the illustrated embodiment, the platform 37 further comprises secondary indicia thereon, wherein the secondary indicia is configured to inform the user of the correct dosage of medication stored within the container in the event of an emergency. In the illustrated embodiment, the secondary indicia reads “Take One Only”.

The container 12 comprises a bottom 13 affixed the platform 37 and an open end 15, wherein a sidewall 14 extends therebetween. In the illustrated embodiment, the sidewall 14 tapers outwardly from the bottom 13 to the open end 15, such that a bowl shaped container 12 is formed. Alternate shapes and configurations of containers are also contemplated, such as cylindrical, square, and the like. In the illustrated embodiment, the container 12 further comprises a cap 33 having a handle 35 thereon, wherein the handle 35 is configured to provide the user with a gripping surface such that the cap 33 can be easily removed from the container. In this way, the container 12 can be sealed to prevent tampering with the contents thereof.

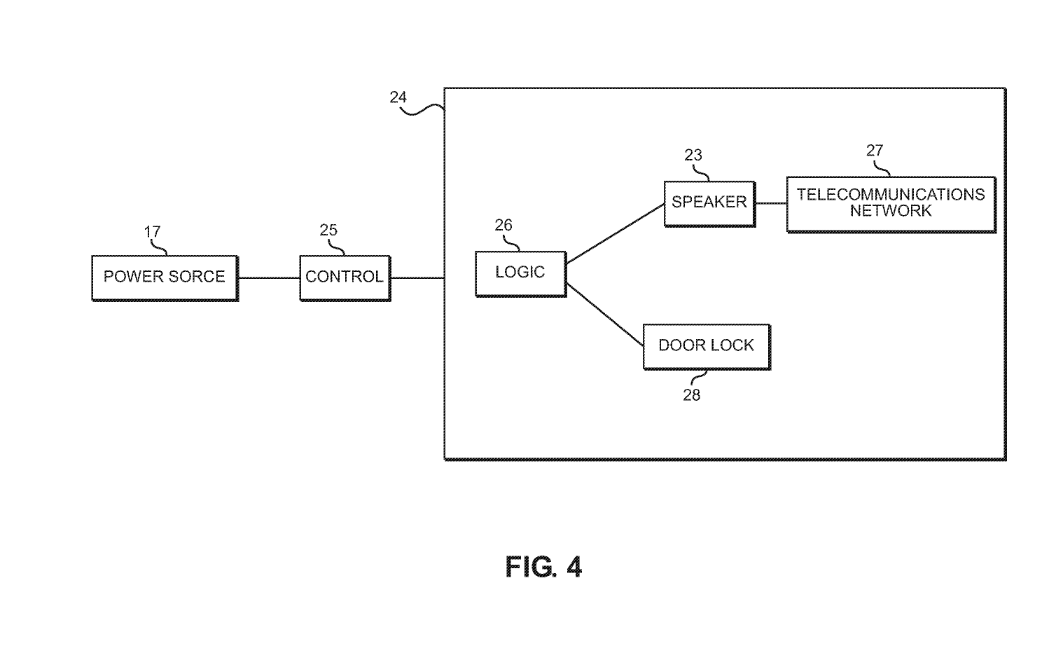

The housing 11 further comprises a speaker 23 thereon, wherein the speaker 23 is operably connected to a microprocessor (as shown in FIG. 4, 24) disposed within the housing 11. The speaker 23 is configured to emit and receive audio signals therethrough, such that a user can engage in two-way telecommunication through the speaker 23. A control 25 is disposed on the housing 11, wherein the control 25 is configured to activate the microprocessor when actuated. In the illustrated embodiment, the control 25 comprises a pull-cord operably connected to a lever, such that the lever is actuated when force is applied to the pull-cord. However, in alternate embodiments, the control 25 can comprise a button, switch, or the like. In the illustrated embodiment, the control 25 further comprises a light 29 thereon, wherein the light 29 is configured to illuminate the control 25 to increase visibility thereof. In this way, a user is ensured that the control 25 is visible in low-light conditions, should the user experience an emergency situation at that time. In some embodiments, the light 29 is configured to emit a Blaze Orange light.

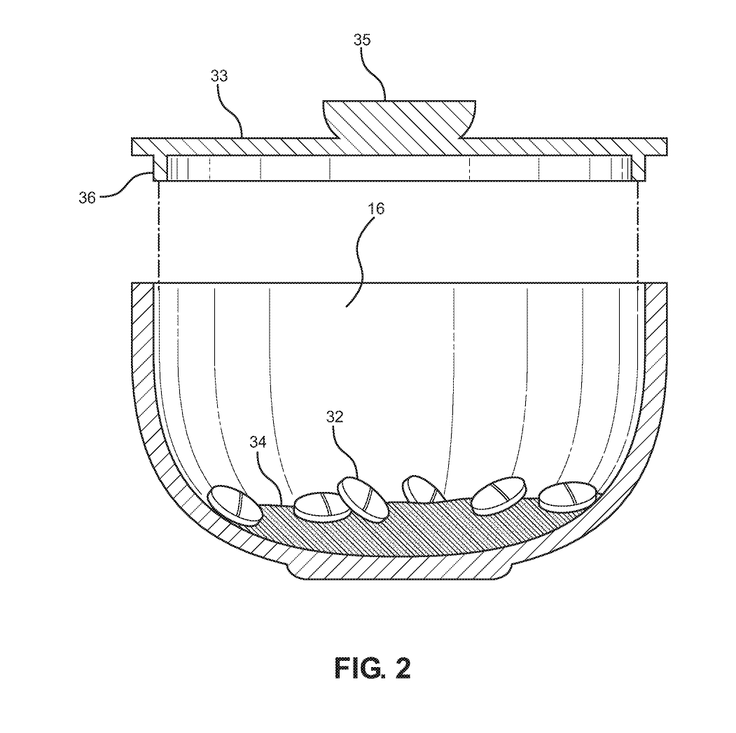

Referring now to FIG. 2, there is shown a cross-sectional view of the container of an embodiment of the emergency responder alert system. In the illustrated embodiment, the container further comprises an interior volume 16. The interior volume 16 is configured to receive at least one pill 32 therein. In some embodiments, the pill 32 comprises an uncoated 325 mg aspirin. In this way, the user can take one pill 32 as indicated by the secondary indicia to dilate blood vessels such that vascular damage due to cardiac arrest can be minimized. In the illustrated embodiment, the interior volume 16 further comprises a desiccant 34 affixed to a lower side of the interior volume 16, such that moisture accumulation within the interior volume 16 does not damage the pills 32. In this way, the desiccant 34 prevents damage to the pills 32, thereby prolonging the efficacy thereof, such that a user does not need to regularly replace the pills 32.

In the illustrated embodiment, the container further comprises a cap 33, wherein the cap 33 includes a handle 35 thereon. The handle 35 is configured to provide a gripping surface to the user, allowing the cap 33 to be easily removed from the container such that the user can access the interior volume 16 at the time of an emergency. In the illustrated embodiment, the cap 33 further comprises a lip 36 extending perpendicularly from a lower surface thereof, wherein the lip 36 is configured to removably secure within the open end of the container via friction fit. In this way, the cap 33 can be removably secured to the container such that movement or jostling thereof does not displace the cap 33, thereby exposing the pills 32 to excess moisture. In some embodiments, the lip 36 further comprises a seal thereabout, wherein the seal is configured to prevent fluid transfer between the environment and the interior volume 16, such that the interior volume 16 is both airtight and watertight.

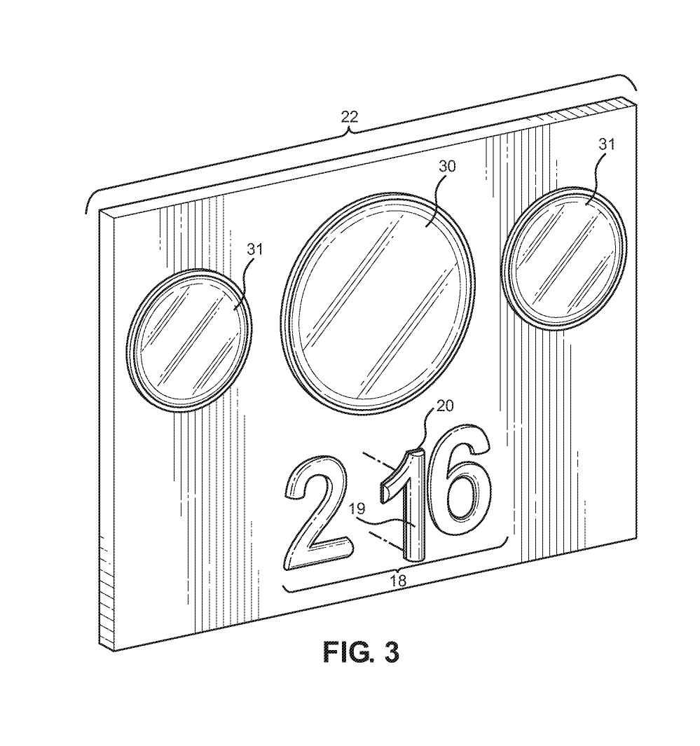

Referring now to FIG. 3, there is shown a perspective view of the lights and indicia of an embodiment of the emergency responder alert system. The emergency responder alert system further comprises a set of first indicia 18, wherein the first indicia 18 is configured to be removably secured to a surface, such as an external wall of a dwelling. The first indicia 18 are configured to indicate the location of the user's home, allowing emergency personnel to easily identify and locate the user. In this way, the emergency personnel can arrive as soon as possible, as the first indicia 18 minimize the risk of the emergency personnel passing by the user's home. In the illustrated embodiment, the first indicia 18 further comprises a reflective front surface 19, wherein the front surface 19 is convex. The convex shape of the front surface creates a larger surface area, thereby allowing more light to reflect therefrom, increasing visibility of the first indicia 18. The first indicia 18 further comprise a fastener disposed on a rear surface 20 thereof, wherein the fastener is configured to removably secure the first indicia 18 to a support surface, such as an exterior wall of a home. In some embodiments, the fastener comprises an adhesive having a cover removably secured thereon, the cover configured to protect the adhesive until the user desires to secure the first indicia 18 to a surface.

The emergency responder alert system further comprises a light source 22 configured to emit light upon the first indicia 18. In this way, the light source 22 provides light to reflect from the front surface 19 of the first indicia 18 during periods of low light. The light source 22 is further configured to illuminate the surrounding area, further indicating the location of the user's home. In some embodiments, the light source 22 comprises high-power sodium lights, such that the light emitted is bright yellow, further standing out from the surrounding area. In the illustrated embodiment, the light source 22 comprises a central light 30 and a pair of opposing lateral lights 31, wherein the central light 30 comprises a larger diameter than that of the pair of lateral lights 31. In this way, the light source 22 is noticeable from a roadway, such that the emergency personnel can easily identify the location of the emergency. In some embodiments, the light source 22 is configured to strobe to increase visibility. The light source 22 can comprise a variety of forms, including but not limited to lights flush with the support surface, globe lighting configured to emit light in a sphere around the light source 22, and the like. In the illustrated embodiment, the first indicia 18 is directly secured to the light source 22, allowing the user to easily install the first indicia 18 at an optimal distance from the light source 22, thereby ensuring maximum reflectivity is achieved.

Referring now to FIG. 4, there is shown a schematic view of an embodiment of the emergency responder alert system. The emergency responder alert system further comprises a power source 17 configured to provide power to the microprocessor 24. In some embodiments, the power source 17 comprises a battery, however, in alternate embodiments, the power source can comprise a direct connection to a home power line, a solar panel, or a combination of one or more of the above. In this way, power can be provided to the microprocessor 24 consistently, even in the case of power outages, low light, and the like. This ensures that the user can always make use of the emergency responder alert system, as an emergency can arise at any moment.

The microprocessor 24 further comprises a logic 26 thereon, wherein the logic 26 is configured to simultaneously activate several subsystems when the control 25 is actuated. When the control 25 is actuated, the logic 26 is configured to activate the speaker 23 to initiate two-way telecommunications with emergency services, such that emergency personnel can be dispatched to the user's location. Additionally, the logic 26 can then contact a third-party, such as a neighbor or family member, that the user selected to be programmed into the microprocessor 24, wherein the third-party will visit the user to wait with them for emergency personnel to arrive. In some embodiments, the third-party is contacted via the telecommunications network, however alternate methods of contact are contemplated, such as text messaging, email notification, and the like. The logic 26 is further configured to actuate a door lock mechanism 28 in operable communication with the microprocessor 24, such that when a signal is received by the door lock mechanism 28, the door lock mechanism 28 selectively moves between a locked position and an unlocked position. In this way, the entry and exit to the user's home is unlocked, allowing emergency personnel to enter the user's home without additional action by the user, ensuring that prompt first aid is received. Furthermore, in some embodiments, the microprocessor 24 is in operable communication with the light source, such that the logic 26 is configured to send a signal to activate the light source when the control is actuated 25. This causes the light source to emit light onto the indicia and out into the surrounding area, thereby increasing visibility of the location of the user's home. In this way, the risk of emergency personnel passing by the user's home is minimized.

In one exemplary use, when the user experiences chest pain or other indicators of cardiac arrest, the user can move to the housing and actuate the control. The user is then placed in contact with emergency services such that emergency personnel can be dispatched to the user's home. The third-party is also contacted and informed that the user is suffering a cardiac arrest, such that the third-party can wait with the user and provide emotional support thereto. When the control is actuated, the light source and door lock are actuated, illuminating the indicia to increase visibility of the user's home and unlocking the entrance to the user's home, respectively. While in contact with emergency services, the user can open the container and ingest a single aspirin therein, such that the aspirin can dilate the blood vessels to minimize the damage thereto while waiting for emergency personnel to arrive to administer additional treatment. In this way, the user can minimize damage caused by a cardiac arrest while efficiently and easily contacting emergency services to come to provide aid.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. An emergency responder alert system, comprising:
a housing having a container affixed thereto;
wherein the container comprises a bottom, a sidewall, and an open end defining an interior volume;
at least one first indicia, wherein each of the first indicia comprises a front side having a reflective surface and a rear side having a fastener thereon, such that the rear side is removably securable to a surface;
a light source configured to illuminate the first indicia;
a speaker disposed on the housing, wherein the speaker operably connected to a microprocessor disposed within the housing;
a control disposed on the housing, wherein the control is operably connected to the microprocessor;
wherein the microprocessor comprises a logic thereon, such that when the control is actuated, the logic is configured to:
contact emergency services, wherein a user can engage in two-way communication with emergency services through the speaker;
actuate a door lock mechanism in wireless communication with the microprocessor, such that the door lock mechanism disengages from a door;
illuminate the light source;
send an alert to a predetermined third-party.

2. The emergency responder alert system of claim 1 further comprising a power source, wherein the power source is configured to provide power to the housing.

3. The emergency responder alert system of claim 2, wherein the power source comprises a battery.

4. The emergency responder alert system of claim 2, wherein the power source comprises a home power line.

5. The emergency responder alert system of claim 2, wherein the power source comprises a solar panel.

6. The emergency responder alert system of claim 1, wherein the control comprises a light thereon, the light configured to illuminate the control.

7. The emergency responder alert system of claim 1, wherein the control comprises a pull-cord.

8. The emergency responder alert system of claim 1, wherein the light source comprises a central light, and a pair of lateral lights, wherein the central light comprises a diameter greater than a diameter of each of the pair of lateral lights.

9. The emergency responder alert system of claim 1, wherein the light source is configured to strobe.

10. The emergency responder alert system of claim 1, further comprising at least one 325 mg uncoated aspirin disposed within the interior volume.

11. The emergency responder alert system of claim 9, wherein the container further comprises secondary indicia thereon, the secondary indicia configured to instruct the user to take one 325 mg uncoated aspirin.

12. The emergency responder alert system of claim 1, further comprising a cap configured to cover the open end.

13. The emergency responder alert system of claim 1, further comprising a desiccant affixed to the bottom of the container, within the interior volume.

14. The emergency responder alert system of claim 1, wherein the container tapers outward from the bottom towards the open end.

15. The emergency responder alert system of claim 1, wherein the front surface comprises a convex shape.

16. The emergency responder alert system of claim 12, wherein the cap further comprises handle thereon.

17. The emergency responder alert system of claim 12, wherein the cap further comprises a lip extending perpendicularly away from a lower surface of the cap, the lip configured to removably secure within the open end via friction fit.

18. The emergency responder alert system of claim 1, wherein the container is affixed to a platform extending from the housing.
